(19) United States
(12) Patent Application Publication
(10) Pub. No.: US 2010/0112689 A1
(43) Pub. Date: May 6, 2010

(54) METHOD OF OBTAINING VIABLE HUMAN CELLS, INCLUDING HEPATIC STEM/PROGENITOR CELLS

(75) Inventors: John W. Ludlow, Carrboro, NC (US); Mark E. Furth, Chapel Hill, NC (US); Andrew T. Bruce, Holly Springs, NC (US); Lola M. Reid, Chapel Hill, NC (US); Robert L. Susick, JR., Chapel Hill, NC (US)

Correspondence Address: FOLEY AND LARDNER LLP;SUITE 500, 3000 K STREET NW, WASHINGTON, DC, 20007, US

(73) Assignees: University of North Carolina at Chapel Hill; Vesta Therapeutics, Inc.

(21) Appl. No.: 12/465,404

(22) Filed: May 13, 2009

Related U.S. Application Data
(63) Continuation of application No. 10/620,433, filed on Jul. 17, 2003, now abandoned.
(60) Provisional application No. 60/396,629, filed on Jul. 19, 2002.

(51) Int. Cl. C12N 5/071

(52) U.S. Cl. 435/370

(57) ABSTRACT

The present invention is directed toward a method for obtaining from whole liver or a resection thereof a population of cells comprising viable, functional liver cells enriched in hepatocytes and hepatocyte stem/progenitor cells, compositions thereof, and uses therefore. Compositions include a composition of liver cells enriched in hepatocytes and hepatocyte stem/progenitor cells and a pharmaceutical composition thereof. Uses include treatment of liver diseases, regeneration of liver, toxicity testing, and liver assist devices.

### 1. CROSS REFERENCE TO RELATED APPLICATIONS

This application is a continuation of U.S. patent application Ser. No. 10/620,433, filed Jul. 17, 2003, which claims priority to U.S. Provisional Patent Application No. 60/396,629, filed Jul. 19, 2002, the entire disclosures of which are incorporated herein by reference.

### 2. BACKGROUND

The normal liver has the ability to regenerate itself by repairing or replacing injured tissue. Despite this protection, once a critical mass of liver cells has died through disease or damage, the liver can fail, leading to illness and death. Liver failure is a serious health problem. Each year, there are an estimated 300,000 hospitalizations and 30,000 deaths in the United States due to chronic liver diseases. Currently, the only cure for many of these liver diseases is a liver transplant. However, only about 5,000 donor livers become available each year in the United States. As of May 2002, approximately 18,000 patients are on the liver transplant waiting list, an increase of more than 100% over the last four years and up from 1,700 ten years ago. Furthermore, approximately 100,000 adults who presently suffer from severe cirrhosis and other forms of chronic liver failure in the United States could become candidates for a transplant.

As a result of the shortage of donor organs, potential liver transplant patients must wait for a donor liver to become available, often for years. Currently, whole organ liver transplantation procedures require a donor who has undergone brain death, but whose heart is still beating. This occurs only in approximately one to two percent of hospital deaths, severely limiting the potential donor pool. Clearly, the vast majority of patients with liver diseases cannot rely on organ transplantation as a solution. There is an urgent need for new technologies to support patients with damaged livers.

The regenerative capacity of the liver suggests that liver cell transplantation might offer a valuable alternative to orthotopic transplantation of whole livers. Donor liver cells infused into a patient with liver disease may be able to colonize the recipient's liver (and/or spleen, if infused into that organ) and restore function. However, the potential of mature hepatocytes to survive for extended periods and to expand in numbers after transplantation remains uncertain.

Conventional wisdom holds that all mature adult liver cells (hepatocytes) are capable of dividing many times, thus allowing the organ to regenerate after injury. However, there is increasing evidence that most hepatocytes actually have a limited capacity to proliferate, while a small percentage of liver cells show properties of stem or progenitor cells that can multiply very extensively and give rise to mature hepatocytes.

Hepatic stem/progenitor cells are a population of immature cells that are committed to the liver lineage, but do not yet express most mature liver cell functions. However, they can both proliferate extensively and give rise to fully differentiated daughter cells that do provide liver function. Studies in rodent models demonstrated the existence of stem/progenitor cells in fetal and adult liver that are at least bipotential; that is, their progeny include two cell types, namely, hepatocytes and bile duct cells. In the adult liver the stem/progenitor cells have been shown to participate in liver regeneration and to extensively repopulate host livers following certain types of liver injury in which the recipient's hepatocytes have an impaired ability to proliferate and/or increase in size through endoduplication.

Over the past 30 years, a significant body of scientific literature has accumulated demonstrating the ability of infused hepatocytes to engraft in host tissue, survive, proliferate, function and participate in the regenerative process. Transplantation of hepatocytes into the spleen or liver has been shown to correct inherited defects in metabolism in numerous models, to completely repopulate a host liver under conditions where the host liver cells have a reduced life-span (as in the FAH-deficient mouse model), to provide hepatic function during acute liver failure induced by a variety of insults, and to improve liver function and prolong survival in CCl4-induced models of cirrhosis.

Case studies and case reports in the literature describe the administration of hepatocytes to over 40 patients with a variety of acute and chronic, inherited and acquired liver diseases. Data from a number of these reports suggest that the cells did indeed engraft, survive and function for up to several months. In one study, the synthetic capabilities of the liver showed improvement four to six months post transplant as evidenced by improved albumin levels and prothrombin time. One of the best published reports demonstrating engraftment and function of transplanted hepatocytes involves a 10-year-old girl with Crigler-Najjar Syndrome, an inherited disease in which the individual is deficient in the enzyme UDP glucuronosyltransferase, which conjugates bilirubin, leading to severe jaundice. Fox et al., I. J., “Treatment of the Crigler-Najjar Syndrome Type I with hepatocyte transplantation,” New England Journal of Medicine, (1998) 338:1422-1426. For 18 months post transplant, this individual experienced significant increases in excretion of conjugated bilirubin in her bile, increased enzyme activity in her liver biopsies and a reduced need for UV light phototherapy. However, these prior experiments with transplanted hepatocytes have resulted in only transient benefit. The limited proliferative ability of hepatocytes necessarily limits the effective life span of treatment with hepatocytes, alone.

It has now been discovered that the aforementioned problems in prior attempts to treat liver diseases are overcome by using populations of cells of the present invention, which are enriched in viable, functional liver cells, including hepatic stem/progenitor cells. The extensive proliferative capacity of the cells of the present invention supports maximal tissue regeneration and lowers the required dose of cells for successful transplantation. The presence of stem/progenitor cells also offers increased effective time span of liver cell therapy due to their improved ability, relative to hepatocytes, to survive, proliferate, function and participate in the regenerative process.

If liver cell therapy is to become a commercial reality and a viable treatment option for a significant number of patients, an adequate supply of liver tissue must be established. It has been discovered that cells obtained by the methods of the present invention may be derived from the livers of certain organ donors, which are not suitable for whole organ transplant or, because of time/transport constraints, cannot be used in a timely fashion. Viable, functional liver cells can be isolated by the method of the present invention from livers, which, by conventional guidelines, are not suitable for orthotopic transplantation or for the preparation of large numbers of mature hepatocytes for cell transplantation. Most importantly, the purification of human liver cell populations, including stem/progenitor cells, by the method of the present invention promises to dramatically expand the donor pool for liver cell therapy. Moreover, apparently because of the relative resistance of stem/progenitor cells to ischemic injury, it has been found that these cells can be obtained by the present method from many asystolic (i.e., non-beating-heart) donors.

The isolation method of the present invention insures that viable, functional liver cells, including hepatic stem/progenitor cells, from the donor liver are included in the cryopreserved mixture of cells. This process isolates a proportionately higher viable liver cell suspension from donated whole human livers, or resections thereof (compared to crude liver preparations), and eliminates dead cells and debris without overly depleting, it at all, the population of small hepatic stem/progenitor cells. The cell population obtained may contain greater than 80% of cells viable before cryopreservation, greater than 70% of cells viable after thawing, and greater than 75% of the cells are hepatocytes.

In contrast, known hepatocyte isolation methods use low speed centrifugation through Percoll to enrich for live hepatocytes (found in the pellet post centrifugation). Although this method is very efficient at isolating large viable hepatocytes, it results in significant depletion of hepatic stem/progenitor cells.

The present invention addresses the aforementioned needs and advances the state of liver cell transplantation or cell therapy by providing a pharmaceutical quality liver cell transplantation or cell therapy product and methods for obtaining highly viable, functional liver cell populations, including hepatic stem/progenitor cells, which are not previously obtainable by conventional methods. A liver cell transplantation or cell therapy product of the present invention consists of a well-characterized mixture of liver cells containing hepatic stem/progenitor cells, as well as other cell types found in the liver.

### 3. SUMMARY OF THE INVENTION

The present invention is directed to a process for obtaining a population of cells enriched in viable human liver cells, including hepatic stem/progenitor cells, comprising: digesting a whole human liver or resection thereof with a proteolytic enzyme preparation to provide a digested whole human liver or resection thereof; dissociating the digested whole human liver or resection thereof to provide a suspension of cells; adjusting the density of the medium in which the cells are suspended whereby at least two bands of cells separated by a density barrier are obtained upon centrifugation, at least one band of the at least two bands being of a lower density than another band of the at least two bands; and collecting the at least one band of lower density to obtain a population of cells enriched in viable human liver cells, including hepatic stem/progenitor cells. Other embodiments of the present invention include, but are not limited to, populations of cells also having functional hepatocytes, functional biliary cells, functional hemopoietic cells, or combinations thereof.

A further embodiment of the present invention provides that the liver or resection thereof may be obtained from beating heart or asystolic neonatal, pediatric, juvenile, or adult donors. In particular, cells may be obtained by the method of the present invention from donor liver that has been subjected to a period of warm ischemia or has been obtained from an asystolic donor.

The present invention is further directed to a composition comprising a population of liver cells enriched in viable, functional liver cells, which population of cells comprise functional hepatocytes and hepatic stem/progenitor cells. In a particular embodiment of the invention, the enriched population of cells is enriched in hepatic stem/progenitor cells having a diameter ranging from about 9 to about 13 microns and which are positive for the expression of EP-CAM (also referred to as GA733-2, C017-1A, EGP40, KS1-4, KSA), CD133, or both.

In a further embodiment, the present invention is directed to a composition comprising a population of liver cells enriched, relative to a crude suspension of cells obtained from liver, in viable, functional hepatocytes and hepatic stem/progenitor cells. An even further embodiment further comprises biliary cells. It has been found that the biliary cells of the cell populations of the present invention are positive for expression of cytokeratin-19 (CK19) and are negative for expression of albumin.

The present invention is also directed to a method of treating liver disease comprising administering an effective amount of a population of cells enriched in viable, functional liver cells, including hepatic stem/progenitor cells. Various modes of administration are contemplated by the present method including, but not limited to, introduction through a splenic artery or portal vein, directly into the liver pulp, under the liver capsule, or directly into the spleen.

In another embodiment, the present invention is directed to a pharmaceutical composition comprising a population of liver cells enriched in viable, functional liver cells, including hepatic stem/progenitor cells and a pharmaceutically acceptable carrier. In a further embodiment, the pharmaceutically acceptable carrier may include a cryopreservative, such as HYPOTHERMOSOL™.

In an even further embodiment, the present invention is directed to a method of conducting in vitro toxicity testing comprising exposing to a test agent a population of liver cells enriched in viable, functional liver cells, including hepatic stem/progenitor cells, and observing at least one effect, if any, of the test agent on the population of liver cells (e.g., on cell viability, cell function, or both). The present invention also contemplates a method of conducting in vitro drug metabolism studies comprising exposing a population of liver cells enriched in viable, functional liver cells, including hepatic stem/progenitor cells, to a test agent, and observing at least one change, if any, involving the test agent after a predetermined test period. The at least one change may include, but is not limited to, a change in the structure, concentration, or both of the test agent.

Another embodiment of the present invention is directed to a liver assist device comprising a housing harboring a population of human liver cells enriched in viable, functional liver cells, including hepatic stem/progenitor cells. The liver cells may comprise human liver cells or porcine liver cells.

The present invention is also directed to a method for treating errors of gene expression comprising introducing into a population of human liver cells, including viable, functional hepatic stem/progenitor cells a functional copy of a gene to provide a transformed population, and introducing into a patient's liver, which patient is in need of the functional copy of the gene, at least a portion of the transformed population. A composition of the present invention useful in the aforementioned method is another embodiment of the present invention.

Other methods provided by the present invention include methods of enhancing the regeneration of an injured or diseased liver, methods of conducting testing for efficacious agents for treating liver infections, methods of producing a protein of interest, and methods of producing a vaccine of interest.

In the method of testing for efficacious agents for treating liver infections, a population of human liver cells is infected with an infectious agent of interest. Thereafter, the infected population is exposed to a predetermined amount of test agent, and the effects, if any, of the exposure on the infected population. In the method of producing a protein of interest, a functional gene encoding the protein of interest is introduced into a population of liver cells including hepatic stem/progenitor cells. The resulting population of cells is then incubated under conditions effective for transcription, translation, and optionally post-translational modification to take place, and thereafter the protein of interest is harvested. Vaccine production is also contemplated whereby a recombinant virus or virion particle is introduced into a population of the cells of the invention, which virus or virion particle is capable of infecting at least some members of the population of cells causing the infected members to express an antigen such that an immune response is elicited from a subject seeking to be immunized against future exposure to an infectious agent associated with the antigen upon introduction of the infected members of the population into the subject.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); Applications and Products 2001: Density Gradient Media (Axis-Shield PoC AS, Oslo, Norway, 2001).

Other features and advantages of the present invention will become apparent from the following detailed description of the invention, taken in conjunction with the accompanying drawings which illustrate by way of example the principles of the invention.

### 4. BRIEF DESCRIPTION OF THE DRAWINGS

FIG. 1 shows a Coulter Counter sizing profile for the novel OptiPrep™ fractionation method, which reveals 2 peaks of cells: those that we have designated relatively “small” (generally ranging from about 9-13 uM) and relatively large (generally ranging from about 18-22 uM). The “small” cell population contain stem/progenitor cells, as these cells are approximately 10 uM in size.

FIG. 2 shows a Coulter Counter sizing profile for the standard (conventional) Percoll method, indicating that the relative abundance of larger cells (18-22 uM) is greater in the Percoll pellet (100×g) than the corresponding supernate (300×g).

FIG. 3 shows the results of FACS analyses following immunostaining with antibody specific for human EP-CAM, revealing that the Percoll pellet (100×g) contains 6-fold less EP-CAM positive-staining cells (left-hand panel, 0.12% of the population (+) for EP-CAM) than the starting material (right-hand panel, 0.76% of the population (+) for EP-CAM).

FIG. 4 shows the results of FACS analyses following immunostaining with antibody specific for human EP-CAM, revealing that the OptiPrep™ fractionation does not appear to affect the overall abundance of the EP-CAM positive-staining population (3.07% and 3.06% of the population staining positive for EP-CAM in the fractionated and unfractionated samples, respectively).

FIG. 5 shows graphs indicating the relative populations of EP-CAM positive cells found in the cell isolation of the present invention (left frame) compared to the supernate of the standard method (center frame) and the pellet of the standard method (right frame). Results are from a nine-month-old donor.

FIG. 6 shows graphs indicating the relative populations of EP-CAM positive cells found in the cell isolation of the present invention (left frame) compared to the supernate of the standard method (center frame) and the pellet of the standard method (right frame). Results are from a 3-year-old donor.

FIG. 7 shows graphs demonstrating enrichment for EP-CAM positive cells after immunoselection.

FIG. 8 shows graphs demonstrating enrichment for EP-CAM positive cells after immunoselection.

FIG. 9 shows photomicrographs of colonies grown from a single cell under various staining conditions, demonstrating that cells isolated by the method of the present invention are hepatic stem/progenitor cells.

FIG. 10 shows photomicrographs of colonies grown from a single cell under various staining conditions, demonstrating that cells isolated by the method of the present invention are hepatic stem/progenitor cells.

FIG. 11 shows a photomicrograph of human hepatocytes obtained by the present invention on microcarrier beads in NOD-SCID mice.

FIG. 12 shows a photomicrograph taken at a lower power than the photomicrograph of FIG. 11 to visualize a large island of hepatocytes that have become vascularized by the host as evidenced by the red blood cells.

FIG. 13 shows evidence that the hepatic stem/progenitor cells of the present invention are able to develop into hepatocytes and are expressing a mature phenotype, demonstrating the positive staining for glycogen in their cytoplasm. Note the apparent organization of the cells into cords.

FIG. 14 shows three hepatocytes obtained by the present invention attached to a microcarrier injected into a host. The black box is drawn around the interface between two adjacent cells. A blow-up of that area shows structures, microvilli, indicative of biliary canaliculi, another mature hepatocyte marker.

FIG. 15 shows a photomicrograph demonstrating engraftment of the cryopreserved human liver cells of the present invention into the livers of NOD-SCID mice. At two hours post transplant, human cells are clearly visible by in situ hybridization in the portal veins and hepatic sinusoids. The cells have not yet passed from the vascular space into the hepatic parenchyma.

FIG. 16 shows a photomicrograph demonstrating engraftment of the cryopreserved human liver cells of the present invention into the livers of NOD-SCID mice. At 40 days post transplant, human cells not only remain in the liver, but have engrafted into the hepatic cell plate becoming fully integrated into the hepatic parenchyma.

FIG. 17A is a flow chart that describes the manufacturing process of the human liver.

FIG. 17B (continued) is a flow chart that describes the manufacturing process of the human liver.

### 5. DETAILED DESCRIPTION OF THE INVENTION

The present invention is directed, in part, to a process for obtaining a population of cells enriched in viable, functional hepatocytes and hepatic stem/progenitor cells. Another, related, aspect of the invention is the identification of hepatic stem/progenitor cells. The embodiments of the present invention described below will impart the important advances made in the identification and isolation of hepatic stem/progenitor cells from adult human liver.

Several cell surface proteins are identified that are expressed by hepatic stem/progenitor cells isolated from human fetal liver. It is found that the same surface antigens are expressed by a small percentage of cells in neonatal, pediatric, and adult human livers. Magnetic cell sorting technology is utilized to greatly enrich for cells expressing one of the surface antigens. The cells isolated by this approach are, on average, much smaller in size than mature hepatocytes, in contrast to previous studies of rodent hepatic stem/progenitor cells which identified a class of large (larger than mature parenchymal cells), acidophilic hepatic cells as liver reserve cells (U.S. Pat. No. 5,559,022). Furthermore, the vast majority of the cells also express a second antigen characteristic of the fetal hepatic stem/progenitors cells. When cultured under conditions that stringently select for the growth of rodent hepatic stem/progenitors, and restrict the growth of more mature hepatic cells, the sorted adult human cells show enhanced growth potential. Most tellingly, analysis of colonies grown from single cells in the sorted population demonstrate the expression of proteins characteristic of both the hepatocyte and bile duct lineages, as anticipated for bipotential hepatic stem/progenitor cells.

It is salient in the present invention that cells expressing the characteristic surface antigens remain present in livers (from non-beating-heart donors), which have suffered several hours of severe oxygen deprivation prior to harvest. In fact, the hepatic stem/progenitor cells seem considerably more resistant to ischemia than mature hepatocytes. Furthermore, although total liver cell preparations from the asystolic donors generally contain greatly elevated numbers of cells associated with tissue damage and inflammatory responses, it still remains feasible to highly enrich for viable, functional liver cells, including hepatic stem/progenitor cells, by the methods of the present invention. In preferred embodiments of the invention, immunoselection and magnetic sorting techniques are utilized to further isolate or remove selected cell types obtained from liver.

The methods of the present invention which are employed to enrich for the viable, functional human liver cells can be applied directly to total liver cell preparations or those prepared from resections of liver. The procedure is rapid, gives favorable cell yields and viability, and can be scaled to process tens of billions of cells. The isolated cells are readily cryopreserved and retain their viability when thawed.

The present invention demonstrates that viable liver cells can be isolated postmortem from a variety of liver sources, including the livers of non-beating-heart donors, whose livers cannot be used for whole organ transplant. Because the liver cell populations of the present invention can be obtained from asystolic donors, the present invention will dramatically expand the pool of donor organs, which are suitable for use in liver cell transplantation or cell therapy. Table 1 summarizes yields from beating heart and asystolic donors.

TABLE 1
Beating Heart Donors

Post-Digestion Yield

| | Total Cells (×10⁹) | Viability (%) |
|---|---|---|
| Range: | 7.7-81.7 | 25-74 |
| Mean: | 32.7 | 57 |

Post-Processing Yield

| | Total Cells (×10⁹) | Viability (%) |
|---|---|---|
| Range: | 1.2-30.7 | 76-99 |
| Mean: | 15.0 | 86 |

Asystolic Donors

Post-Digestion Yield

| | Total Cells (×10⁹) | Viability (%) |
|---|---|---|
| Range: | 0.1-26.2 | 11-51 |
| Mean: | 10.7 | 34 |

Post-Processing Yield

| | Total Cells (×10⁹) | Viability (%) |
|---|---|---|
| Range: | 0.01-11.0 | 64-99 |
| Mean: | 5.0 | 86 |

### 5.1 Method of Isolation of the Present Invention and Comparison to a Standard (Conventional) Method

Cells are isolated from whole donor livers or resections thereof by perfusing the tissue with Liberase™, a purified form of collagenase, and collecting the resulting cell suspension. Two methods are examined to separate live cells from dead ones. In the novel method of the present invention, an aliquot of hepatic cell suspension is mixed with an equal volume of a solution of iodixanol (OptiPrep™, 60% iodixanol in water, Axis-Shield, Noway), and centrifuged at 2000 rpm (approximately 500×g) in a Cobe 2991™ cell washer (available from Blood Component Technology, Lakewood, Colo.) for 15 minutes at room temperature, as follows.

To a sterile 500 ml bottle, add 208.5 ml of OptiPrep™, 291.5 ml of RPMI-1640 without phenol red. This results in a 25% solution of iodixanol having a density of 1.12. After calculating the volume of cells based on weight, add enough RPMI-1640 without phenol red to bring the final volume up to 250 ml, with a total cell number of 10×10⁹ (40×10⁶ cells/ml). Add 250 ml of 25% iodixanol and agitate gently to mix well. Gravity feed the resulting iodixanol cell solution into a COBE 2991™ cell washer-processing bag. Layer 100 ml of RPMI-1640 without phenol red on top of the iodixanol cell solution using a peristaltic pump at a rate of 20 ml/min while the bag is spinning. Centrifuge at 2000 rpm (approximately 500×g) for a total of 15 minutes. The resulting hepatic cell band at the interface between the iodixanol cell solution and the RPMI-1640 without phenol red is recovered separately from the pelleted material.

In separate experiments leading to the conditions described above, the densities of starting materials and those of selected centrifugation bands, including one designated a “Umix” band, a “gradients content” band and the pellet, are determined. It is found that the band of interest, the “Umix” band, has a density of 1.0607. This density value is less than that found for starting material (1.0792), “gradient contents” band (1.0792) and the pellet (1.1061). It is then determined that an 11.59% solution of iodixanol is needed to provide a gradient directly over which the cells of interest would settle after centrifugation.

In the standard method, an aliquot of hepatic cell suspension is mixed with isotonic Percoll (Sigma, Mo.) to a final concentration of 22.5% Percoll. Following centrifugation at 100×g for 5 minutes at 4° C., in a Sorvall RC3B centrifuge, the pellet is recovered. It should be noted that in the teachings of conventional methods, the supernate is discarded because of the conventional belief that the supernatant contains cells of lower viability and that it contains generally more cellular debris. For comparative purposes, the supernate is recovered, diluted 5-fold and centrifuged at 300×g for 5 minutes at 4° C., and the resulting pellet recovered.

Trypan blue exclusion reveals that the 100×g Percoll pellet is enriched for viable cells (70-90% range) compared to the Percoll supernate (40-60% range). In contrast, for the OptiPrep™ gradient of the present invention, the upper-most band of cells is enriched for viable cells (80-90%) compared to the pelleted material (generally less than 20%). Size analyses using the Coulter Counter reveals enrichment for larger cells (18-22 uM in diameter) in the Percoll pellet, compared with the Percoll supernate, which contains a larger population of cells in the 9-13 uM in diameter range than the pellet. The upper-most band from the OptiPrep™ gradient contains both 18-22 uM in diameter and 9-13 uM in diameter cells. Size distribution determination for the OptiPrep™ pellet is problematic owing to the large amount of debris. Fluorescence Activated Cell Sorting (FACS) analyses following EP-CAM immunostaining of these cells indicates that sedimentation through Percoll results in depletion of EP-CAM positive-staining cells; these positive cells remain behind in the Percoll supernate. The upper-most band of the OptiPrep™ gradient has a population of EP-CAM positive cells similar to that of the Percoll supernate. Colony forming assays reveal virtually no formation of colonies for cells found in the Percoll pellet, while the Percoll supernate has a comparable level of colony forming ability as the upper-most band of the OptiPrep™ gradient. This colony-forming ability correlates with EP-CAM positive staining, as enriching for EP-CAM positive cells also enriches for colony forming ability of the cell preparation.

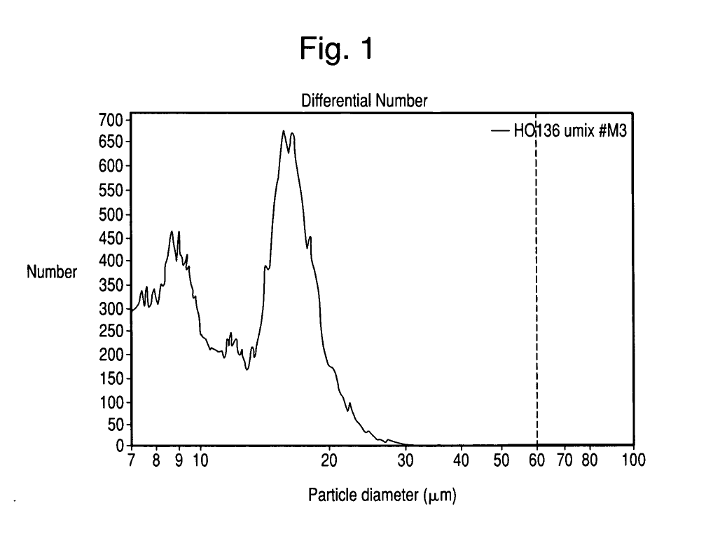

As illustrated in FIG. 1, a Coulter Counter sizing profile for the novel OptiPrep™ fractionation method reveals 2 peaks of cells: those that we have designated relatively small (generally ranging 9-13 uM in diameter) and large (generally ranging 18-22 uM in diameter). The small cell population contain stem/progenitor cells, as these cells are approximately 10 uM in size. The relative abundance of these 2 populations of cells varies depending upon the donor liver, as does the average size in microns of the peak population.

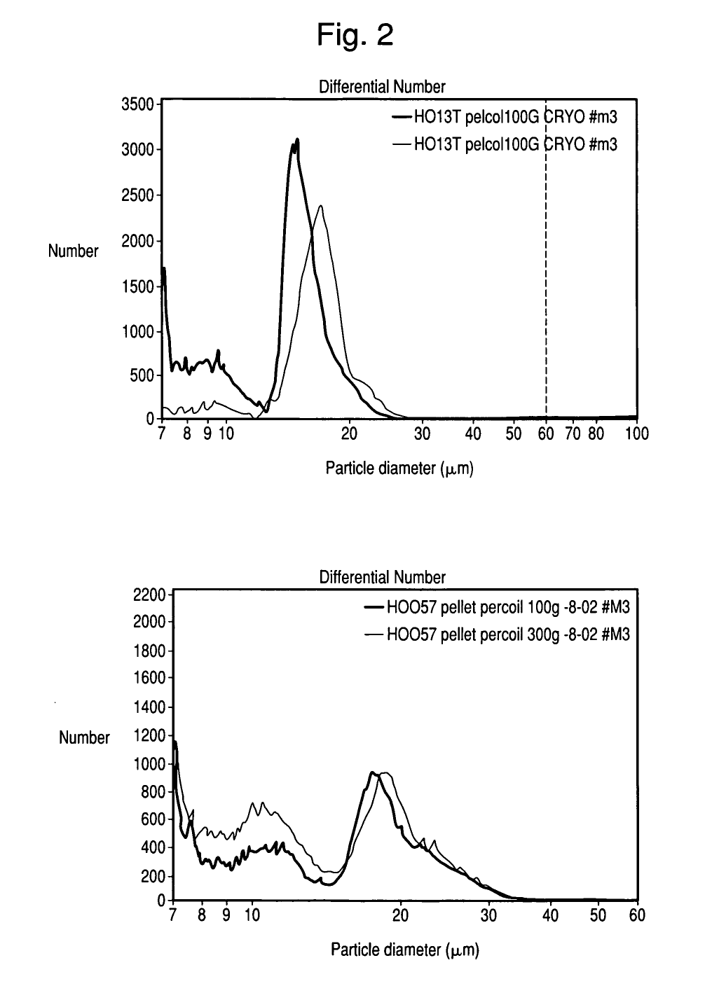

FIG. 2 illustrates that, following the standard Percoll method, the relative abundance of larger cells (18-22 uM in diameter) is greater in the Percoll pellet (100×g) than the corresponding supernate (300×g).

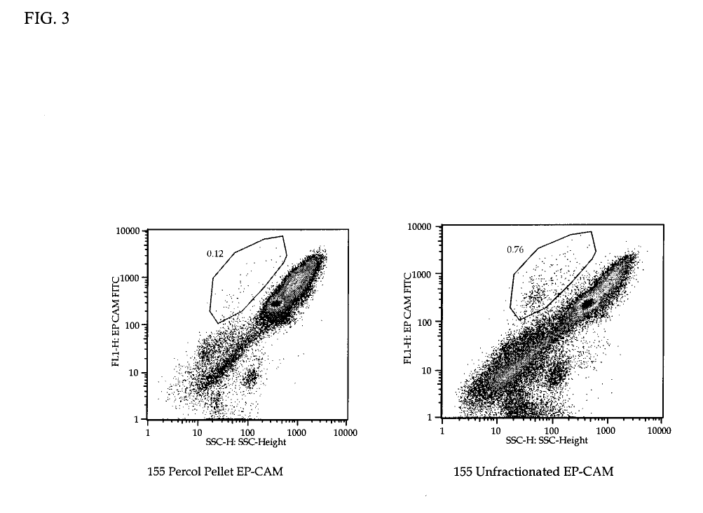

FIG. 3 shows the results of FACS analyses following immunostaining with antibody specific for human EP-CAM, revealing that the Percoll pellet (100×g) contained 5-fold less EP-CAM positive-staining cells (left-hand panel, 0.12% of the population (+) for EP-CAM) than the starting material (right-hand panel, 0.76% of the population (+) for EP-CAM).

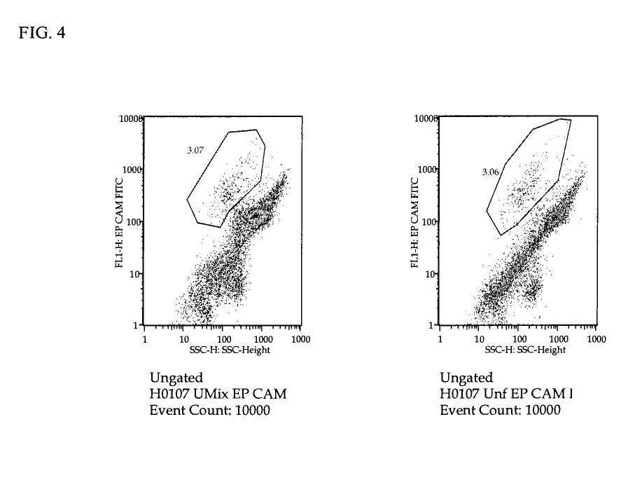

In contrast, as shown in FIG. 4, OptiPrep™ fractionation does not appear to affect the overall abundance of the EP-CAM positive-staining population (3.07% and 3.06% of the population staining positive for EP-CAM in the fractionated, and unfractionated samples, respectively).

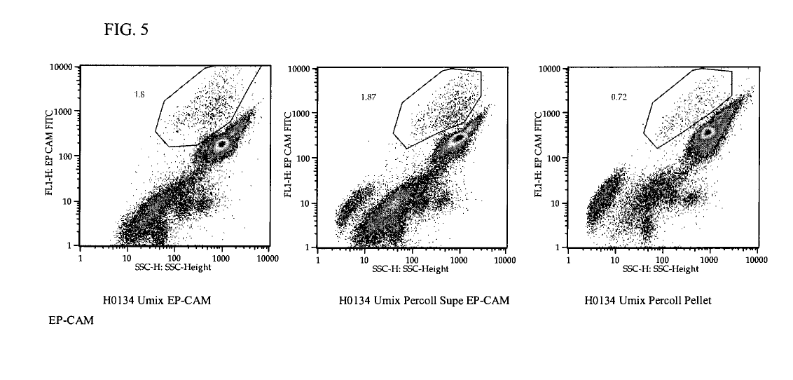

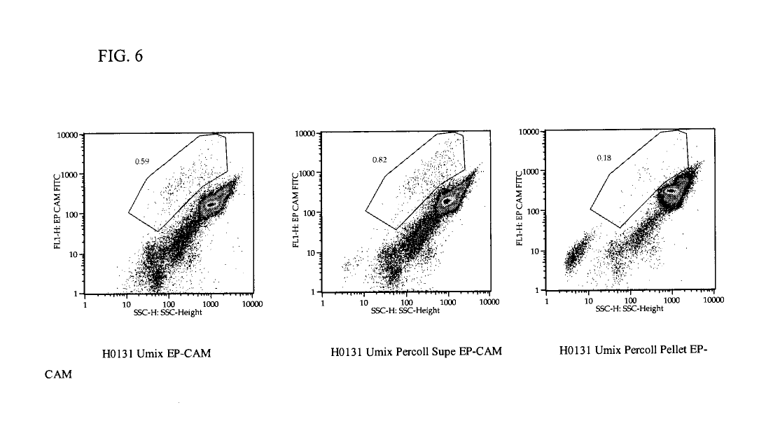

Using OptiPrep™ fractionated cells (the upper-most band) as the starting material, in experiments using 2 different donor livers, we also demonstrate that the EP-CAM positive immunostaining cells remain in the supernate following the standard Percoll method. As illustrated in FIGS. 5 and 6, the population of EP-CAM positive cells is comparable for the OptiPrep™ fractionated cell starting material and the Percoll supernate, while the Percoll pellet is depleted 2-5 fold for these positive cells.

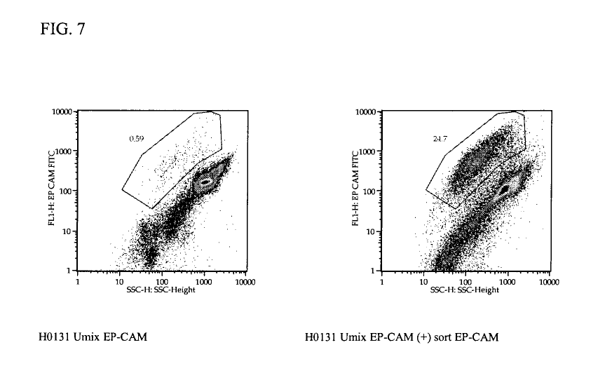

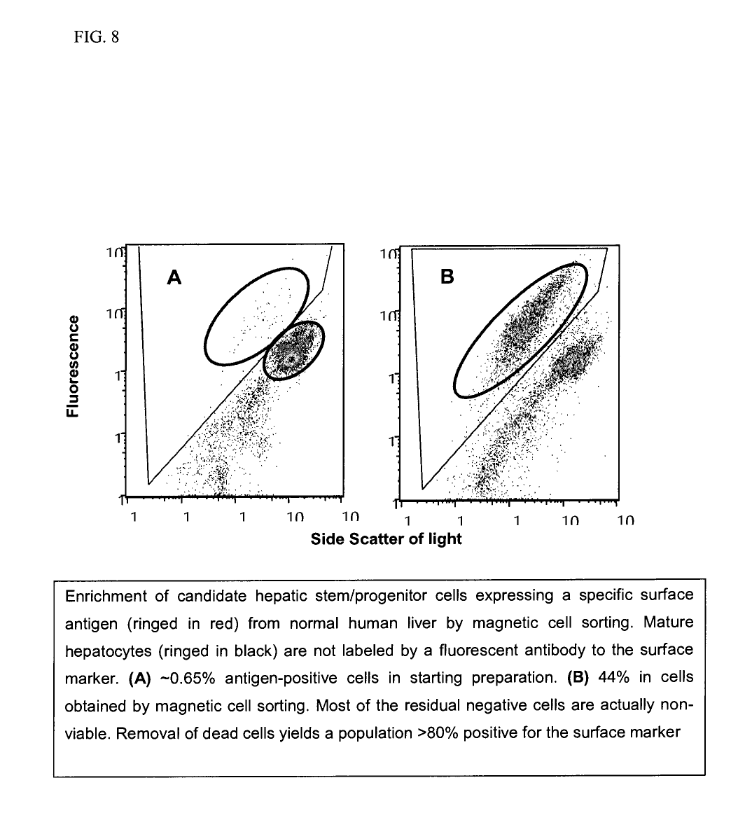

As a biological test for stem/progenitor cell presence in these cell preparations following OptiPrep™ fractionation and Percoll density gradient centrifugation, 20,000 live cells/well are plated onto STO feeder layers, maintained in hormonally-defined medium, and scored for colony formation after a 2-week incubation. To further support our contention that EP-CAM immunoreactivity corresponds to stem/progenitor cell presence, we rationalize that if we increase the population of EP-CAM cells, we should increase the number of colony-formers in that population. Towards this goal, we enrich by immunoselection for these cells and include them in our assay. As shown in FIGS. 7 and 8, we enrich for EP-CAM positive cells 40-fold (0.59% of the starting population is EP-CAM positive, but after immunoselection, 24.7% of the population is positive for this marker).

Table 2 shows that we indeed enrich for colony formation when we enrich for EP-CAM positive-cells. In addition, OptiPrep™ fractionated cells and the Percoll supernate both contain colony forming cells, while the Percoll pellet is lacking such cells.

TABLE 2

| | Total | Average | | | | | | |
|---|---|---|---|---|---|---|---|---|
| Fraction | Colonies | Colonies/Well | Well 1 | Well 2 | Well 3 | Well 4 | Well 5 | Well 6 |
| OptiPrep ™ | 9 | 1.5 | 1 | 0 | 2 | 2 | 2 | 2 |
| fraction | | | | | | | | |
| EP-CAM⁺ | 43 | 7.2 | 7 | 7 | 11 | 12 | 5 | 1 |
| enrich | | | | | | | | |
| Percoll Pellet | 1 | 0.2 | 0 | 0 | 1 | 0 | 0 | 0 |
| Percoll | 20 | 3.3 | 3 | 1 | 2 | 2 | 7 | 5 |
| Supernate | | | | | | | | |

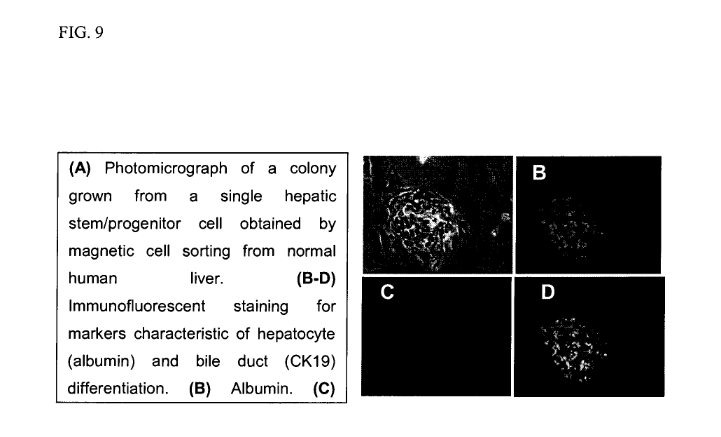

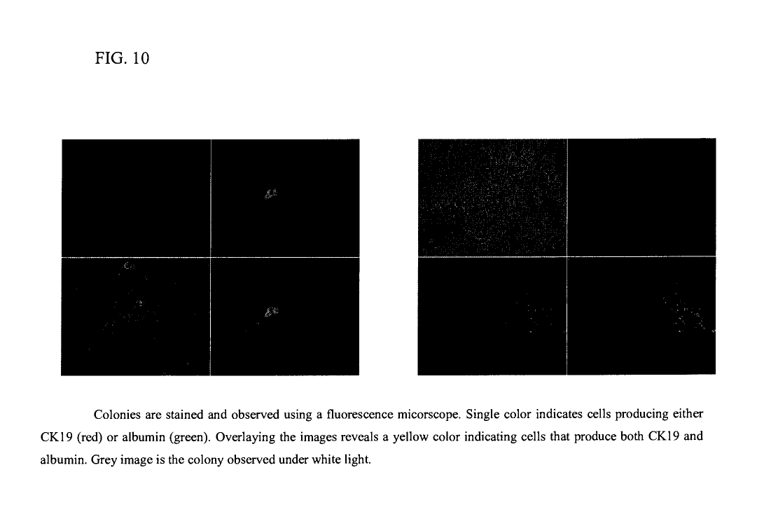

To ensure that these colonies arise from a single cell, limiting dilutions are performed on EP-CAM positive-enriched cells, and wells containing an average of 1 cell are immunostained with antibody to human albumin, indicative of a parenchymal cell, and CK19, which reacts with biliary cells. As shown in FIGS. 9 and 10, the presence of both of these types of cells in a single colony is supporting evidence for the bi-potentiality of the cell giving rise to that colony. Such bipotentiality is the operational definition of a stem/progenitor cell.

These data clearly indicate that the novel OptiPrep™ fractionation method of the present invention separates live from dead cells while retaining viable hepatic stem/progenitor cells in the live fraction. In contrast, the standard method in the field, of centrifugation through a Percoll density gradient, excludes these cells from the pellet. While there are modifications in the conditions under which Percoll density gradients are run, all of these modifications involve short centrifugation times (minutes) and low g-force (50, 70, 88×g). The object of the conventional method appears to be enrichment for larger size, mature, viable hepatocytes. Since the Percoll pellet is used for all subsequent experiments (the supernate is discarded), the field has been consistently using cell preparations depleted of such proliferative stem/progenitor cells. Our novel method will most certainly change the types of experiments performed, and the data generated, to further advance the field. This is particularly true in the area of cell transplantation, where it is deemed crucial to have transplanted cells with the highest proliferative capacity, so as to have the greatest probability of reconstituting liver function.

Immunoenrichment (immunoselection) is merely one means of enriching a population of hepatic stem/progenitor cells of the present invention. Monoclonal antibodies are particularly useful for identifying markers (surface membrane proteins, e.g., receptors) associated with particular cell lineages and/or stages of differentiation. Procedures for separation of the subject stem/progenitor cells may include magnetic separation, using antibody coated magnetic beads, affinity chromatography, and “panning” with antibody attached to a solid matrix, e.g., plate, or other convenient technique. Techniques providing accurate separation include fluorescence activated cell sorting, which can have varying degrees of sophistication, e.g., a plurality of color channels, low angle and obtuse light scattering detecting channels, impedance channels, etc.

Conveniently, the antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the cells.

Cells of the present invention may be preserved by cryopreservation. Typically, isolated cells (as described above) are diluted to a desired concentration in an aqueous mixture of Hypothermosol™ (Biolife Solutions, NY) and subjected to controlled freezing to a desired storage temperature. Frozen cells of the present invention may be stored in liquid nitrogen.

### 5.2 Functionality of Cells of the Present Invention

Once liver cell populations, including hepatic stem/progenitor cells, of the present invention are isolated and cryopreserved, they are characterized by flow cytometry utilizing cell-specific monoclonal or polyclonal antibodies and fluorochrome-conjugated secondary antibodies to quantify cell types present. In addition, the functionality of the cryopreserved cells is assessed across a battery of in vitro and in vivo endpoints.

For example, hepatic stem/progenitor cells of the present invention may be reacted on ice with 100 uL mouse monoclonal IgG polymorphic antibodies to human EP-CAM antigens conjugated to fluorescein isothiocyanate (FITC) (Serotec Inc, UK). Control cells are treated with mouse IgG-FITC alone. The samples are analyzed using an EPICS C flow cytometer (Coulter Electronics, Hialeah, Fla.) tuned to a wavelength of 488 nm with the fluorescence gain adjusted to exclude 98% of the control cells. Windows are established around the various cell populations using the forward light scatter (FLS) vs. side scatter (SS) two parameter histogram and the percentage of positively fluorescent events is determined.

In vitro endpoints include: 7-ethoxycoumarin metabolism, which measures both microsomal cytochrome P-450 dependent phase I oxidation as well as coupled Phase II conjugation reactions; ureagenesis to assess the cells' ability to convert ammonia to urea (an important function lost during liver failure); and proliferation potential.

In vivo we assess the cells' ability to: survive over time; establish and maintain a mature hepatocyte phenotype; and engraft into the liver parenchyma.

The following studies utilize NOD-SCID mice that have a severe combined immune deficiency preventing the animals from rejecting the transplanted human cells. In one study, cells are thawed and incubated, in vitro with microcarriers to which they attach. The microcarriers are subsequently injected into the peritoneal cavity of the mice. One week later, the microcarrier cell conglomerates are harvested from the peritoneal cavity, sectioned and stained for light microscopy. Electron microscopy is also performed.

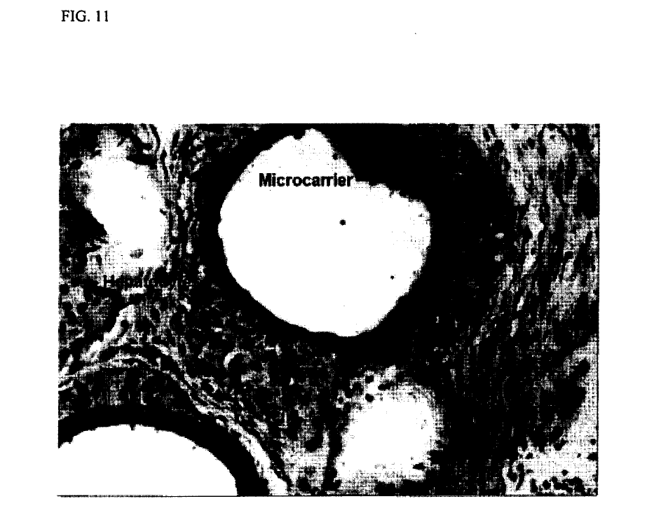

In FIG. 11, a photomicrograph of human hepatocytes on microcarrier beads in NOD-SCID mice, one can clearly see hepatocytes attached to the microcarriers. Note their rounded nuclei and large amount of clear cytoplasm. The right arrow indicates a binucleated cell. Cells on far right are host stromal cells, likely fibrobalsts, from the peritoneal lining of the recipient mouse.

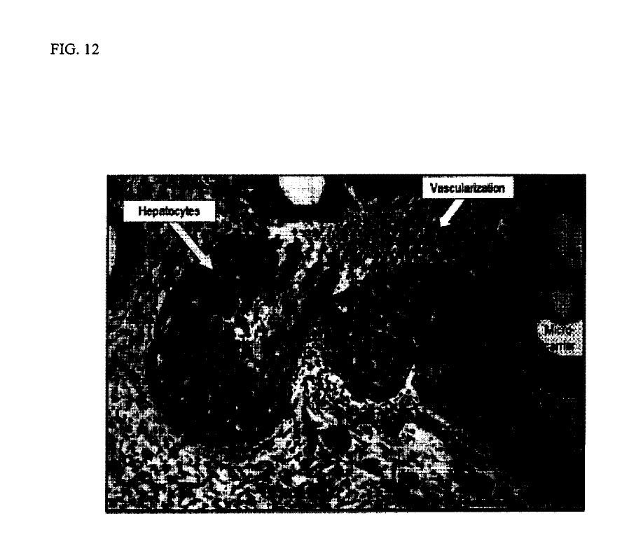

FIG. 12 shows a photomicrograph taken at a lower power to visualize a large island of hepatocytes that have become vascularized by the host as evidenced by the red blood cells. Particularly noteworthy is the apparent organization of the cells into cords or rows of hepatocytes, a structural organization that is readily observed in cross sections of liver tissue.

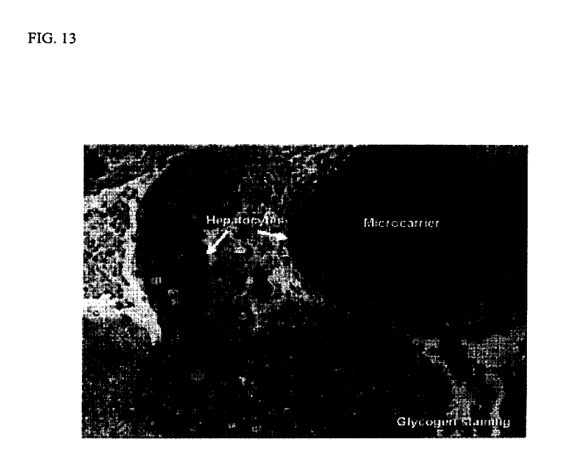

Evidence that the hepatic stem/progenitor cells of the present invention can indeed mature into hepatocytes and are expressing a mature phenotype is provided in FIG. 13, demonstrating the positive staining for glycogen in their cytoplasm. Again, note the apparent organization of the cells into cords.

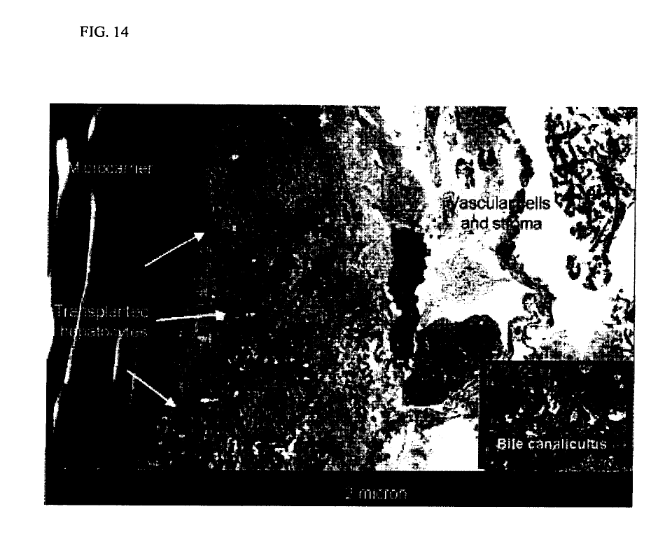

At the electron microscopic level, one can discern in FIG. 14 three hepatocytes attached to this microcarrier. The black box is drawn around the interface between two adjacent cells. A blow-up of that area shows structures, microvilli, indicative of biliary canaliculi, another mature hepatocyte marker.

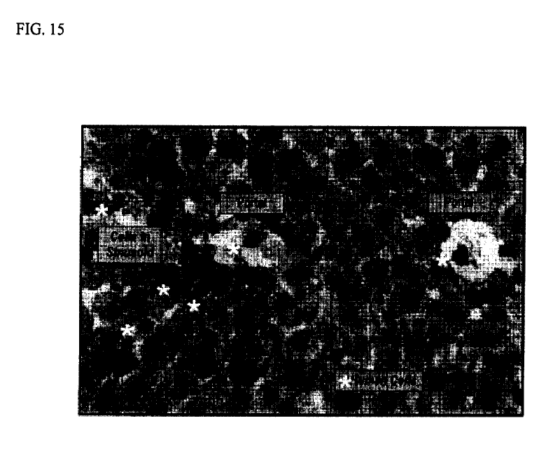

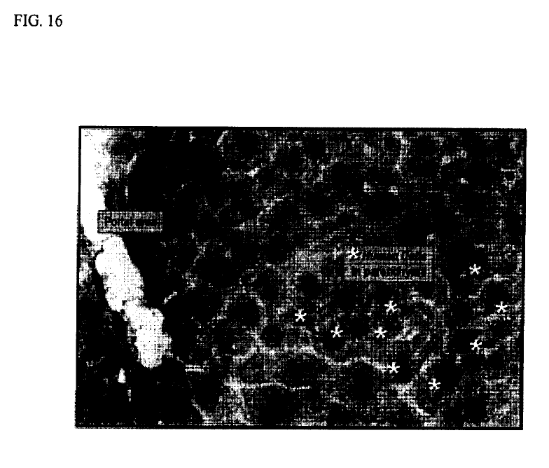

The two photomicrographs shown in FIGS. 15 and 16 demonstrate engraftment of the cryopreserved human liver cells of the present invention into the livers of NOD-SCID mice. In this study, 1 million thawed cells are injected into the spleens of the mice. At various time points after transplantation, animals are euthanized and the presence of human cells determined by in situ hybridization using DNA probes for human centromeres as well as PCR analysis. At two hours post transplant (FIG. 15), human cells are clearly visible by in situ hybridization in the portal veins and hepatic sinusoids. They have not yet passed from the vascular space into the hepatic parenchyma, however.

At 40 days post transplant (FIG. 16), human cells not only remain in the liver, but have engrafted into the hepatic cell plate becoming fully integrated into the hepatic parenchyma.

### 5.3 Liver Cell Transplantation

The target population for treatment with cells of and by the method of the present invention are ambulatory patients with cirrhosis and end-stage liver disease (ESLD) caused by a variety of factors. Patients have a life expectancy without liver transplant of greater than six months but less than two years. Most have been considered for placement on a solid liver transplant list. These patients have experienced one or more complications of their disease, such as abdominal fluid (ascites), bleeding, confusion (hepatic encephalopathy), infections and other problems. They are all given immunosuppression therapy to prevent rejection of transplanted liver cells. The goal of therapy is to delay or obviate the need of a solid liver transplant, to reduce hospitalizations for complications of liver disease and to improve patient quality of life.

Baseline and follow up assessments include routine laboratory and clinical liver function assessments as well as specific quantitative biochemical assessments of the ability of the damaged liver to remove toxins, metabolize drugs and synthesize proteins. Because transplanted liver cells are expected to populate both the liver and spleen, liver cell-specific scans of the spleen are performed periodically to monitor engraftment and proliferation of transplanted liver cells. Transplanted cells release soluble antigens that are specific to the donor cells. These soluble antigens, which can be measured in the blood, are monitored as further evidence for viability and function of transplanted cells.

Two weeks before admission to the hospital for cell transplantation, patients are seen in the clinic by the investigator. The investigator obtains informed consent and begins baseline assessments, including ABO blood typing. Patient's blood type must be compatible with donor blood cells in solid liver or liver cell transplantation. Two days before hospital admission, immunosuppression therapy is begun. Cryopreserved cells are shipped to the hospital where they remain frozen until just before transplantation.

In the evening prior to cell transplantation, the patient enters the hospital. The following morning, the patient is transferred to the invasive radiology suite where he/she receives conscious sedation. A catheter is placed in the patient's femoral artery (in the groin) and advanced into the splenic artery. Donor liver cells are thawed, diluted and delivered preferably through a syringe into the splenic artery catheter. Administration time varies, depending on dose, from five to approximately 30 minutes. The catheter is removed and the patient transferred back to his/her room for follow-up care. The patient is discharged from the hospital eight hours after the procedure.

Hepatocyte and hepatic stem/progenitor cell transplantation of the present invention may be used to effect replacement of liver function by injecting a quantity of viable, functional liver cells including hepatocytes and/or hepatic stem/progenitor cells (contained within a transplant medium such as saline) into an appropriate anatomic site where the liver cells, including hepatocytes and/or and hepatic stem/progenitor cells are allowed to implant within an extracellular matrix and express differentiated liver functions, including hepatocyte functions. Depending upon the quantity of liver cells, including hepatocytes and/or and hepatic stem/progenitor cells, so transplanted, different degrees of liver function deficiencies may be corrected by replacement of liver function with the cellular transplants. Cellular transplantation of hepatocytes and/or and hepatic stem/progenitor cells is most advantageous, however, in treating liver disease caused by genetic defects resulting in the absence or decreased function of a single enzyme or other protein product. Such diseases include, for example, the hyperlipidemias and alpha-antitrypsin deficiency. Other diseases of the liver treatable with the present invention include hepatitis, cirrhosis, inborn errors of metabolism, acute liver failure, acute liver infections, acute chemical toxicity, chronic liver failure, cholangiocitis, biliary cirrhosis, Alagille syndrome, alpha 1-antitrypsin deficiency, autoimmune hepatitis, biliary atresia, cancer of the liver, cystic disease of the liver, fatty liver, galactosemia, gallstones, Gilbert's syndrome, hemochromatosis, hepatitis A, hepatitis B, hepatitis C, poryphyria, primary sclerosing cholangitis, Reye's syndrome, sarcoidosis, tyrosinemia, type 1 glycogen storage disease, and Wilson's disease.

In order to perform the transplantation procedure, an injection site is selected to transplant liver cells into the liver parenchyma. In one technique for accomplishing this, the injection site is the patient's spleen. After computation of the spleen's location coordinates, the injector is positioned to inject a transplant medium containing liver cells, including hepatocytes and/or and hepatic stem/progenitor cells, into the spleen. The transferred cells then migrate via the splenic vein into the liver parenchyma (See Gupta et al., Seminars in Liver Disease 12, 321 (1992)). In another technique, branches of the portal vein are imaged by, for example, CAT scanning of the abdomen after injection of a radiopaque contrast medium. The location coordinates of the portal branches feeding the separate lobes of the liver may then be used to inject the transplant medium into a portal branch and thus infuse a specific liver lobe with liver cells. Such selective infusion allows continued portal blood flow through the other liver.

Alternatively, liver cells, including hepatocytes, biliary cells, and/or and hepatic stem/progenitor cells, of the present invention may be injected or infused directly into the liver pulp, through the splenic vein or portal vein, or beneath the liver capsule.

Suitable methods of administering the cells of the present invention to subjects, particularly human subjects, are described in detail herein, including injection or implantation of the cells into target sites in the subjects, or the cells of the invention can be inserted into a delivery device which facilitates introduction by injection or implantation of the cells into the subjects. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. The liver cells, including hepatic stem/progenitor cells of the invention can be inserted into such a delivery device, e.g., a syringe, in different forms. For example, the cells can be suspended in a solution or embedded in a support matrix when contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating viable, functional liver cells as described herein in a pharmaceutically acceptable carrier or diluent and, as required, other ingredients enumerated above, followed by filtered sterilization.

Support matrices in which the viable, functional cells can be incorporated or embedded include matrices which are recipient-compatible and which degrade into products that are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. See e.g., U.S. Pat. No. 4,298,002 and U.S. Pat. No. 5,308,701. These matrices provide support and protection for the liver cells in vivo and are, therefore, the preferred form in which the liver cells are introduced into the recipient subjects.

### 5.4 Gene Therapy of the Present Invention

Gene therapy clinical trial results have in general been disappointing for both physicians and patients, often because of the inability to obtain sustained gene expression of the target gene. Progenitor cells, because of their extensive expansion potential, represent a promising cell population to produce continued gene expression. The gene therapy of the present invention, in one embodiment, will be achieved by inserting an exogenous gene into the liver progenitor cells and transplanting these cells into the patient. Logical target disorders are diseases resulting from the inability of the patient's liver cells to properly make an important protein, such as the missing LDL receptors in hypercholesterolemia and clotting factors in hemophilia.

A major impediment in the current attempts to achieve stable integration of foreign genes in eukaryotic host cells of different organs is the inability of most of these cells to proliferate in vitro. This is particularly problematic for attempts to insert exogenous genes in liver cells, since hepatocytes do not normally undergo cell division in vitro. Recently, gene transfer studies are performed using hepatocytes isolated from Watanabe heritable hyperlipidemic rabbits, which are widely used as an animal model for familial hypercholesterolemia in humans. Like their human counterparts, the Watanabe rabbit cells contain a genetic deficiency in low density lipoprotein (LDL) receptor, leading to high levels of cholesterol in the circulation and increased incidence of premature coronary artery disease (Wilson et al., 1990, Proc. Natl. Acad. Sci. USA 87:8437). Rabbit hepatocytes are infected with recombinant viruses carrying a functional LDL receptor gene, and shown to cause a temporary amelioration of hyperlipidemia in the genetically deficient rabbits following transplantation. It is believed that the success rate of this form of therapy can be further augmented if the gene of interest can achieve more stable integration into a population of recipient cells, which is capable of substantial cell division. Since the hepatic stem/progenitor cells of the present invention proliferate in vitro, especially for longer time periods in the co-culture system described herein, these cells may be ideal candidates as recipients for the introduction of exogenous genes in culture.

A variety of inborn errors of metabolism are caused by inherited genetic deficiency in liver cells. These diseases may be treated by transplantation of liver cells of the present invention carrying functional copies of the correct genes. In brief, this procedure involves isolation of liver cells, including hepatic stem/progenitor cells, of the present invention from patients afflicted with a particular deficiency, transfer of functional genes into these cells to correct the genetic defect by conventional gene transfer technologies, confirmation of stable integration and expression of the desired gene products, and transplantation of the cells into the same or other patients' own livers for reconstitution. This approach is particularly applicable in situations where a single gene defect is responsible for the disease and the defective gene has been identified and molecularly cloned; however, it is not limited only to these conditions. In addition to gene therapy in an autologous setting, hepatic stem/progenitor cells of the present invention carrying functional genes may also be transplanted into allogeneic HLA-matched individuals. Examples of target genes and their related liver diseases that are amenable to this form of therapy include, but are not limited to, the LDL receptor gene in familial hypercholesterolemia, the clotting factor genes for factors VIII and IX in hemophilia, the alpha 1-antitrypsin gene in emphysema, the phenylalanine hydroxylase gene in phenylketonuria, the ornithine transcarbamylase gene in hyperammonemia, and complement protein genes in various forms of complement deficiencies.

The liver is a center of production for many secretory proteins. It is anatomically connected with the circulatory system in such a way that allows a efficient release of various proteins into the bloodstream. Therefore, genes encoding proteins that have systemic effects may be inserted into liver cells of the present invention as opposed to the specific cell types that normally produce them, especially if it is difficult to integrate genes into these cells. For example, a variety of hormone genes or specific antibody genes may be inserted into liver cells of the present invention for the secretion of their gene products into the circulation.

For the practice of the invention, liver cells of the present invention isolated by the procedures described above are used as recipients in gene transfer experiments. The cells may be grown in culture prior to, during, or after introduction of an exogenous gene. In vitro differentiation of these cells may be minimized by the addition of cytokines in a manner similar to the use of leukemia inhibitory factor in hematopoietic stem cell cultures.

For the introduction of exogenous genes into the cultured cells of the present invention, any cloned gene may be transferred using conventional techniques, including, but not limited to, microinjection, transfection and transduction. In addition, if the liver cells express receptors for the asialoglycoprotein, plasmids containing the genes of interest may be conjugated to asialoglycoprotein and added to cells to induce uptake and expression (Wu et al., 1991, J. Biol. Chem. 266:14338). This procedure is more gentle on the recipient cells.

The preferred method of gene transfer utilizes recombinant viruses, such as retroviruses and adenoviruses. For example, when using adenovirus expression vectors, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a nonessential region of the viral genome (e.g., region E1 or E3) will result in a recombinant virus that is viable and capable of expressing the gene product in infected liver reserve cells (e.g., see Logan & Shenk, 1984, Proc. Natl. Acad. Sci. USA 81:3655-3659). Alternatively, the vaccinia virus 7.5K promoter may be used. (e.g., see, Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931). Of particular interest are vectors based on bovine papilloma virus which have the ability to replicate as extrachromosomal elements (Sarver, et al., 1981, Mol. Cell. Biol. 1:486). Shortly after entry of this DNA into cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as, for example, the neo gene. Alternatively, the retroviral genome can be modified for use as a vector capable of introducing and directing the expression of any gene of interest in hepatic stem/progenitor cells of the present invention (Cone & Mulligan, 1984, Proc. Natl. Acad. Sci. USA 81:6349-6353). High level expression may also be achieved using inducible promoters, including, but not limited to, the metallothionine IIA promoter and heat shock promoters.

For long-term, high-yield production of recombinant proteins, stable expression is preferred. Rather than using expression vectors which contain viral origins of replication, viable, functional liver cells, including hepatic stem/progenitor cells, of the present invention can be transformed with a cDNA controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered liver cells may be allowed to grow for 1-2 days in an enriched media, and then are switched to a selective media. A number of selection systems may be used, including but not limited to the herpes simplex virus thymidine kinase (Wigler, et al., 1977, Cell 11:223), hypoxanthineguanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes. Also, antimetabolite resistance can be used as the basis of selection for dhfr, which confers resistance to methotrexate (Wigler, et al., 1980, Proc. Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072; neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes. Recently, additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85:8047); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.).

The liver cells of the present invention that have integrated a particular gene as measured by their expression of its gene product by techniques such as Northern blots and ELISA, may be transplanted, as described above, into the patients from whom the cells are originally derived or into a HLA-matched individual. For HLA-matched allogeneic transplantation, the liver reserve cells may not necessarily require gene transfer prior to transplantation. For instance, liver reserve cells obtained from a donor who possesses a functional gene encoding clotting factor VIII may be used directly by transplantation into a HLA-matched hemophiliac patient. The transplanted cells will presumably multiply and give rise to mature PC performing normal liver functions, including the production of clotting factor VIII.

In addition to using the liver cells of the present invention for correcting liver gene defects, these cells may be used to replenish the liver parenchyma in the case of hepatic cirhosis, as noted above, or they may be engineered against liver specific infectious diseases. For example, uninfected hepatic stem/progenitor cells may be obtained from an early stage hepatitis patient and used as recipients for genes encoding anti-sense RNA that is complementary to critical replication-related genetic elements of a hepatitis virus. The cells may then be transplanted into the patients to control spread of the virus and restore normal liver function.

### 5.5 Genomics and Research Applications

The liver cell technology of the present invention has application as a tool for identifying new drugs and in the drug development and testing process. The liver stem/progenitor cells can be made to grow and differentiate into mature liver cells. Determining gene expression patterns at various stages of the liver lineage provides genomic information for drug discovery. For example, this information can be used to identify new targets for drug discovery programs or to identify proteins performing biological functions that may have applications in therapy.

As a tool for the drug testing and development process, the liver cells and their progeny could be used to assess changes in gene expression patterns caused by drugs being considered for development. The changes in gene expression pattern from potential drugs could be compared with those caused by drugs known to affect the liver. This would allow a pharmaceutical company to screen compounds for their effect on the liver earlier in the development process, saving time and money. The full lineage of liver cells, from progenitors to mature cells, could also be used to test drugs for toxicity to the liver and to study how the drug is metabolized. Currently, pharmaceutical companies have difficulty obtaining a consistent supply of liver cells for toxicity testing. The methods of the present invention answer this need.

### 5.6 Liver Assist Device

The liver stem/progenitor cell technology of the present invention has application in the development of a liver assist device (“LAD”). LAD's are designed to provide treatment for patients with acute liver failure by providing liver function for a short period of time (7 to 30 days) to allow sufficient time for a patient's own liver to recover from failure or to provide a bridge to transplant.

Attempts at clinically useful LADs by others have utilized pig hepatocytes or poorly differentiated liver cells derived from human tumors in a wide variety of bioreactor types. These devices have shown promise, but all utilize cells with limitations that our cells should overcome. The pig hepatocytes, while easily obtained, have severe limitations; e.g., immune reactions to secreted pig proteins, limited lifetime and non-human viruses. The liver tumor cells can easily be grown, but retain only a subset of the functions of normal liver cells and involve safety concerns. Functioning human liver cells from donor organs have not been an alternative due to the scarcity of donor livers. LAD using human liver progenitor cells of the present invention will overcome many of the problems experienced to date. Proteins secreted by these cells will be of human origin so immune reactions should be minimized. The progenitor cells can divide extensively in culture so that cells from one donor liver may be able to supply many LADs. Most importantly, these cells should display the wide range of liver functions necessary for clinical utility.

An example of an LAD suitable for the cells of the present invention is described in International Patent Publication Serial Number PCT US00/15524.

### 5.7 Vaccine Manufacture of the Present Invention

The liver cells of the present invention can also be utilized for the production of vaccines. For example, replication-deficient virus (e.g., a lentivirus—see, Naldini et al. Science 272:263-267, 1996) can be used to infect human liver cells, wherein the virus has been further modified to harbor genes encoding one or more specific protein antigens. The specific protein antigens are chosen depending on the type of immune response desired. Basically, liver cells of the present invention are infected with the recombinant virus. The infected cells then express a protein antigen against which an immune response is mounted. It is expected that the immune response (antibody or cell based) is directed against an infectious agent, such as hepatitis C. Subjects exposed to the infected cells are then protected against the infectious agent. The reader is referred to Brister et al. (see, J. Gen. Virol. 83 (Pt. 2):369-381, 2002) for a description of the use of recombinant Semliki Forest virus coding for hepatitis C non-structural protein to elicit a cellular immune response.

### 6. EXAMPLES

### 6.1. Process Summary

All processing of the liver is performed in a class 100 hood, located in a class 10,000 room, following aseptic techniques and in compliance with good manufacturing processes. All components that contact the liver are purchased as sterile or are assembled and subjected to gas sterilization or autoclaving.

### 6.2. Initial Processing

The liver is received submerged in VIASPAN™ (see, http://www.viaspan.com/viaspan/pdf), triple bagged in a cooler on wet ice. In a biological safety cabinet (BSC), the liver is weighed, and its gross appearance is documented. A sample of the VIASPAN™ is taken for sterility testing. (VIASPAN™ is useful as a hypothermic solution for flushing and storage of organs.) The liver is moved into a sterile bin and soaked in an antibiotic wash (0.1 mg/mL Gentamicin and 5 mg/mL Cefazolin) for 5 minutes. The liver is turned from top to bottom during this procedure to ensure that both sides are soaked.

The liver is lifted and rinsed twice with a total volume of 2 L of sterile normal saline over a bin. The liver is then transferred to another sterile bin. The vena cava is clamped using two sterile, disposable, plastic umbilical cord clamps and the portal vein and/or hepatic artery are cannulated with pre-sterilized cannulae made of plastic reducer/connector of various sizes. A small biopsy (from the leading edge of a lobe) is taken for histologic observation. The liver is transferred to a perfusion tank and perfused with warm (≦37° C.) chelation buffer for 15 minutes at a rate that allows maximal ballooning of the liver (typically 120-240 mL/min). At the end of the perfusion period, the buffer is drained to waste through a drain port located on the bottom of the perfusion tank.

### 6.3. Perfusion and Digestion

The liver is then digested with a perfusate containing LIBERASET™ CI (an enzyme preparation containing collagenase and elastase) for 30 minutes at 28° C.-37° C. At the end of digestion the LIBERASE™-containing buffer is drained, and the liver is perfused with cold serum-containing collection buffer to stop digestion by the enzyme. After the last perfusion, the buffer is drained into the waste container, and the tank is replenished with new serum-containing collection buffer. The liver capsule is serrated using a sterile stainless steel surgical scalpel and the tissue is massaged (for not more than 20 minutes) to facilitate the dissociation of cells. When all cells from the digested tissue appear to have been dissociated into the buffer, the resulting cell suspension is passed through a pre-filter, and a series of 1000, 500, 250 and 150 μm pre-sterilized stainless steel sieves, and then collected into a 4-liter blood bag chilled on ice. The crude cell suspension is sampled for in process testing of viability, concentration, total cell count, yield per gram tissue and sterility.

### 6.4. Downstream Processing

The crude cell suspension is aseptically transferred into an appropriate number of 600 mL blood bags and concentrated by centrifugation at 800×g. The concentrated cell suspension is enriched for live cells by mixing equal volumes of the cell concentrate and an OPTIPREP™ solution (25% Iodixanol) and using the COBE 2991 cell washer. After centrifugation at 2000 rpm for 15 minutes, the desired cell population will move to the top and form a band. The bands are aseptically collected and distributed into an appropriate number of 600 mL blood bags, at a volume preferably not exceeding 200 mL/bag. The volume in the bag is then diluted to 500 mL with RPMI 1640. The bag is centrifuged at 800×g for 10 minutes, and the supernatant is expressed out. The resulting pellet is weighed, and enough RPMI 1640 is added to achieve a final volume of 500 mL, and centrifuged at 800×g for 10 minutes. After the supernatant is removed, the post-wash pellet is weighed, sampled for cell count and viability, and re-suspended in HTS to achieve a concentration of 6×107 cells/mL. If multiple COBE runs are involved due to a large number of cells, the bands collected from each run will be pooled.

### 6.5. Fill and Storage

The cells are then manually filled (at a fill-volume of 1.5 mL) into labeled, 33-mL fluoroplastic cryobags and subsequently mixed with an equal volume of cryobuffer (HTS: DMSO: Human serum 60:20:20) to achieve a final concentration of 3×107 cells/mL, 10% DMSO and 10% human serum. The bags are frozen using a Cryomed programmable freezer, and the frozen cells are stored in vapor nitrogen freezers. At least 24 hours post freezing, samples are pulled from the freezer and shipped to designated testing facilities for release testing.

### 6.6. Process Flow Chart

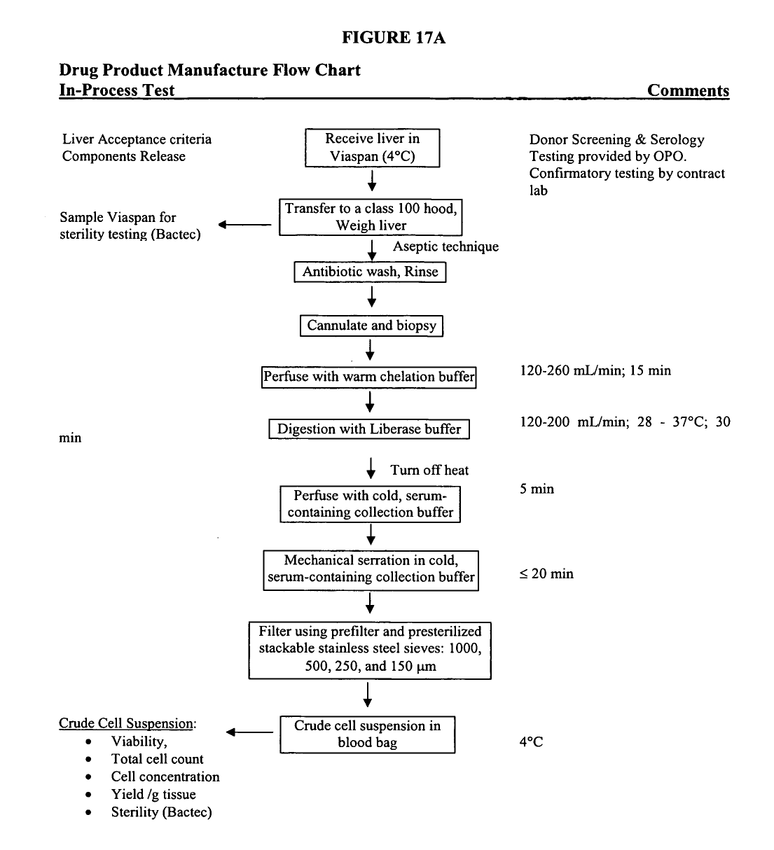

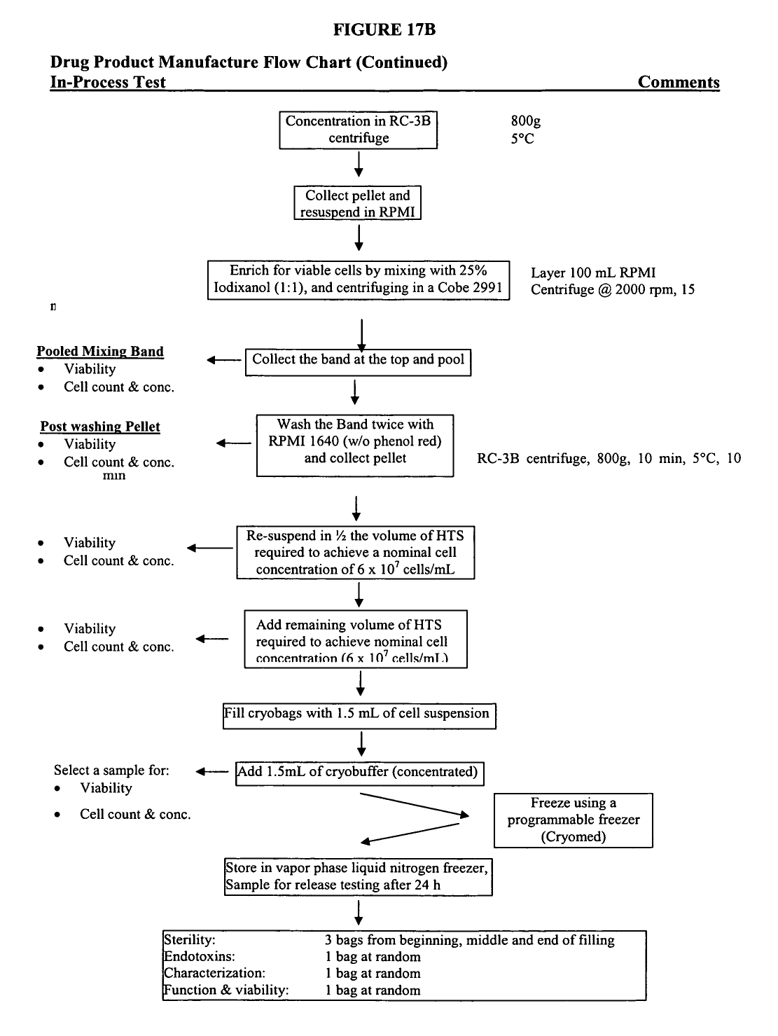

A flow chart that describes the manufacturing process performed is provided in FIGS. 17A and 17B.

### 6.7. Administration at Clinical Sites

Clinical supplies will be shipped to clinical sites in qualified vapor phase liquid nitrogen shippers that maintain a temperature at or below −120° C. The cryobags containing the cell suspension will remain in the shipper until the patient is ready. Before use, the product is removed from the shipper and quickly thawed at 37° C., and placed over ice. The over-wrap is then removed, and, using standard, aseptic hospital procedures, the cell suspension is diluted ten-fold with cold Plasma-Lyte® A in the cryobag prior to administration to the patient. This procedure precludes the need to wash the cells prior to infusion and minimizes the risk of compromising sterility. One embodiment of the invention that will be given to the patient comprises 3×106 cells/mL, and further comprise DMSO (1%), human serum AB (1%), HypoThermosol® (4%-8%) and RPMI without phenol red (0%-4%) in Plasma-Lyte®.

### 6.8. Isolation of Porcine Liver Cells

The procedure of filtering and collecting a cell suspension, as described above, is followed for a sample of porcine liver.

The sample is tested for viability, density and yield. After calculations are made, 10 billion cells are removed. If the density is lower than 25 million cells per mL, the cells are concentrated using either the Sorval RC3B centrifuge, Sorval centritech or the COBE 2991 cell processor. The pellet is resuspended in 250 mL of RPMI 1640 media without phenol red. The cell suspension is transferred to a 600 mL blood bag and an equal volume (250 mL) of 25% Iodixanol (Opti-prep™, see, http://www.nycomed-diagnostics.com/gradmed/optiprep/optil.html) diluted in RPMI 1640 w/o phenol red is added. The two solutions are mixed together thoroughly and kept cold.

The COBE 2991 cell processor is set up using a single processing set. The cell suspension is gravity fed using the red line of the set. Once the doughnut is filled, centrifugation is begun at 2000 rpm for 15 min. As centrifugation begins 100 mL of RPMI 1640 media is layered on top of the gradient using a peristaltic pump at 20 mL/min to act as buffer for the mixing band. After 15 min the top buffer is “decanted” at a speed of 100 mL/min into a waste bag and the top cell band is collected in a collection bag. The pellet is also collected, if desired, for future analysis. The top cell band is placed on ice and sampled for viability and yield. The process is repeated until all the unfractionated porcine hepatocytes are processed. Once all the bands are collected and pooled together, the combined collected cell bands are then washed by diluting in collection buffer and centrifugation at 3000 rpm for 10 min using a Sorval RC3B. The resulting pellet is resuspended in cryo-preservation buffer at a density of 30 million/mL. Aliquots, as needed, are placed in bags and/or vials. The final porcine cell preparations are stored in a controlled-rate freezer over liquid nitrogen.

The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

What is claimed is:

1. A process for obtaining a population of cells enriched in viable human liver cells, including hepatic stem/progenitor cells, comprising:
(a) digesting a whole human liver or resection thereof with a proteolytic enzyme preparation to provide a digested whole human liver or resection thereof;
(b) dissociating the digested whole human liver or resection thereof to obtain a suspension of cells;
(c) suspending the suspension of cells in medium comprising 25% (w/v) iodixanol;
(d) centrifuging the suspension to obtain at least two bands of cells separated by a density barrier; and
(e) collecting the band having density less than 1.0792 to obtain a population of cells enriched in viable human liver cells, including hepatic stem/progenitor cells.

2. The process of claim 1 in which the population of cells enriched in viable human liver cells further includes functional hepatocytes.

3. The process of claim 1 in which the population of cells enriched in viable human liver cells further includes functional biliary cells.

4. The process of claim 1 in which step (a) includes:
(f) perfusing the whole human liver or resection thereof with a chelation buffer;
(g) digesting the whole human liver or resection thereof with an enzyme preparation comprising collagenase and at least one other proteolytic enzyme at approximately 37° C. to provide a digested liver.

5. The process of claim 4 in which the enzyme preparation includes at least one neutral protease.

6. The process of claim 4 in which the enzyme preparation includes elastase.

7. The process of claim 4 in which the enzyme preparation comprises both collegenase and neutral protease.

8. The process of claim 1 in which said dissociation includes mechanical dissociation.

9. The process of claim 8 in which said dissociation includes mechanical dissociation by cutting, raking, combing, or grating the liver.

10. The process of claim 1 in which step (c) includes at least one of:
(h) filtering the cell suspension to remove debris and cell aggregates;
(i) collecting the resulting filtered cell suspension in a first bag;
(j) optionally determining a concentration of cells in the filtered cell suspension;
(k) adjusting, if desired, the concentration of cells to provide a starting cell suspension.

11. The process of claim 1 in which step (d) includes at least one of:
(n) collecting the at least one band into a collection buffer on ice;
(o) determining viability and concentration of cells;
(p) washing the cells by centrifugation and resuspension in a cryopreservation buffer to obtain a final cell suspension;
(q) subjecting the final cell suspension to controlled rate freezing to provide a frozen cell suspension; and
(r) storing the frozen cell suspension in a liquid nitrogen freezer.

12. The process of claim 11 in which said collection buffer comprises RPMI 1640 medium with 10% human or bovine serum.

13. The process of claim 10 in which said filtering includes passing said cell suspension through a filter cartridge.

14. The process of claim 1 in which said medium lacks phenol red.

15. The process of claim 10 in which said centrifugation is carried out for about 15 min at approximately 500×g.

16. The process of claim 11 in which said container includes a collection bag.

17. The process of claim 11 in which the cryopreservation buffer comprises a mixture including Na⁺, K⁺, Ca²⁺, Mg²⁺, Cl⁻, H₂PO₄⁻, HCO₃⁻, HEPES, lactobionate, sucrose, mannitol, glucose, Dextran-40, adenosine, glutathione, or combinations thereof.

18. The process of claim 17 in which the cryopreservation buffer further comprises serum and dimethylsulfoxide.

19. The process of claim 18 in which the mixture, serum and dimethylsulfoxide are present in a ratio of approximately 80:10:10 v/v/v.

20. The process of claim 18 in which the serum comprises human serum, bovine serum, or a combination thereof.

21. The process of claim 1 in which the enriched population of cells includes hepatic progenitor/stem cells having a diameter in the range between 9 and 13 microns and which are positive for expression of EP-CAM, CD133, or both.

22. A process for obtaining an enriched population of viable human liver cells, which population of cells comprises functional hepatocytes and hepatic stem/progenitor cells, comprising:
(a) obtaining a whole human liver or resection thereof from neonatal, pediatric, juvenile, adult, or cadaver donor;
(b) perfusing the whole human liver or resection thereof with a chelation buffer;
(c) digesting the whole human liver or resection thereof with an enzyme preparation to provide a cell suspension;
(d) optionally, mechanically dissociating the whole liver or resection thereof to provide a cell suspension;
(e) optionally, removing debris and cell aggregates;
(f) mixing the cell suspension with an equal volume of iodixanol solution;
(g) subjecting the resulting mixture overlaid with a predetermined volume of culture medium to centrifugation to obtain at least two bands of cells separated by a density barrier, at least one band being of a lower density than another band bands; and
(h) collecting the at least one band of density less than 1.0792.

23. The process of claim 22 in which the enriched population of cells is enriched in hepatic progenitor/stem cells having a diameter in the range between about 9 and about 13 microns and which are positive for expression of EP-CAM, CD133, or both.

24. The process of claim 22 in which the perfusing is carried out with a chelation buffer.

25. The process of claim 22 in which the enzyme preparation comprises collegenase, elastase, or both.

26. The process of claim 22 in which the removing of debris and cell aggregates is carried out by passing the cell suspension through a filter cartridge.

27. The process of claim 22 in which the iodixanol solution is in RPMI 1640 medium.

28. A method of obtaining an enriched population of viable human liver cells, which population of cells comprises functional hepatocytes and hepatic stem/progenitor cells, comprising:
(a) obtaining a whole human liver or resection thereof;
(b) digesting the whole human liver or resection thereof to provide a suspension of liver cells;
(c) mixing an aliquot of the suspension of liver cells with a solution of 25% (w/v) iodixanol;
(d) centrifuging the resulting mixture to obtain at least one band of less than 1.0792 density and enriched for viable cells; and
(e) collecting the at least one band of viable cells.

29. The process of claim 28 in which the density of at least one band is 1.0607.

30. The method according to claim 28 in which the liver is from neonatal, pediatric, juvenile, adult, or cadaver donor.

31. The method of claim 28 in which the digesting is performed with an enzyme preparation comprising collagenase, elastase or a combination thereof.

32. The method of claim 28 in which the solution of iodixanol lacks phenol red.

33. The method of claim 28 further comprising overlaying the resulting mixture of liver cells and solution of iodixanol with a predetermined volume of medium lacking phenol red prior to the centrifuging step.

34. The method of claim 28 in which the centrifuging is performed on a COBE™ 2991 Cell Processor.
